(19) United States
(12) Patent Application Publication
(10) Pub. No.: US 2011/0125241 A1
(43) Pub. Date: May 26, 2011

(54) LEAD INCLUDING COMPOSITE DEVICE FOR ELUTING A STEROID AND AN ANTIMICROBIAL

(75) Inventors: Kenneth E. Cobian, St. Anthony, MN (US); Genevieve L. Gallagher, Mendota Heights, MN (US); Peter M. Seiler, St. Anthony Village, MN (US)

(73) Assignee: Medtronic, Inc., Minneapolis, MN (US)

(21) Appl. No.: 12/625,385

(22) Filed: Nov. 24, 2009

(51) Int. Cl. A61N 1/05; A61M 37/00

(52) U.S. Cl. 607/120

(57) ABSTRACT

A lead may include a lead body including a proximal end and a distal end, an electrode proximate to the distal end of the lead body, and a monolithic controlled release device located proximate to the electrode. According to this aspect of the disclosure, the monolithic controlled release device includes a polymer, a steroid mixed in the polymer, and an antimicrobial mixed in the polymer.

### TECHNICAL FIELD

The disclosure relates to implantable medical devices and, more particularly, to techniques for reducing risk of post-implantation infection.

### BACKGROUND

Implantable medical devices (IMDs) include a variety of devices that provide therapy (such as electrical simulation or drug delivery) to a patient, monitor a physiological parameter of a patient, or both. In providing therapy or monitoring a physiological parameter, the IMD is often coupled to a lead. The lead includes at least one conductor that electrically couples an electrode located on a distal end of the lead to the IMD, which is connected to a proximal end of the lead. The lead may also include a passive or an active fixation element. The fixation element may be located proximate to the electrode, and may assist in reducing or even eliminating lead migration.

### SUMMARY

In one aspect, the disclosure is directed to a lead comprising a lead body including a proximal end and a distal end, an electrode proximate to the distal end of the lead body, and a composite monolithic controlled release device located proximate to the electrode. According to this aspect of the disclosure, the composite monolithic controlled release device includes a polymer, a steroid mixed in the polymer, and an antimicrobial mixed in the polymer.

In another aspect, the disclosure is directed to a system including an implantable medical device and a lead. According to this aspect of the disclosure, the lead includes a lead body including a proximal end and a distal end, an electrode proximate to the distal end of the lead body, and a composite monolithic controlled release device located proximate to the electrode. The composite monolithic controlled release device may include a polymer, a steroid mixed in the polymer, and an antimicrobial mixed in the polymer.

In a further aspect, the disclosure is directed to a method including forming a monolithic controlled release device comprising a polymer, a steroid, and an antimicrobial and assembling the monolithic controlled release device, a lead body, a conductor, and an electrode to form a lead in which the monolithic controlled release device is proximate to the electrode.

In an additional aspect, the disclosure is directed to a method including implanting in a patient a lead comprising a lead body including a proximal end and a distal end, an electrode formed proximate to the distal end, and a monolithic controlled release device (MCRD) located proximate to the electrode. According to the aspect of the disclosure, the MCRD comprises a polymer, a steroid mixed in the polymer and an antimicrobial mixed in the polymer. The method further includes eluting the steroid from the MCRD to a tissue of the patient proximate to the MCRD and eluting the antimicrobial from the MCRD to the tissue of the patient proximate to the MCRD.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example therapy system that may be used to provide cardiac stimulation therapy to a patient, and which includes a lead including a composite monolithic controlled release device.

FIG. 2 is a conceptual diagram illustrating further details of the therapy system of FIG. 1.

FIG. 3 is a conceptual diagram of an example of a lead including an electrode, a composite monolithic controlled release device, and a passive fixation element.

FIGS. 4A-4E are conceptual diagrams of configurations of a composite monolithic controlled release device including a first material and a second material.

FIG. 5 is a conceptual diagram of an example of a lead including an electrode, a composite monolithic controlled release device, and a passive fixation element.

FIG. 6 is a cross-sectional diagram illustrating an example of a lead including an electrode and a composite monolithic controlled release device.

FIG. 7 is a cross-sectional diagram illustrating an example of a lead including an electrode and a composite monolithic controlled release device.

FIG. 8 is a conceptual diagram of an example of a lead including a combination electrode and active fixation element and a composite monolithic controlled release device.

FIG. 9 is a cross-sectional diagram of an example of a lead including a combination electrode and active fixation element and a composite monolithic controlled release device.

### DETAILED DESCRIPTION

In general, the disclosure is directed to a lead that includes a composite monolithic controlled release device (MCRD) that elutes both a steroid and an antimicrobial into a body of a patient after implantation of the lead in the body of the patient. The composite MCRD may be located proximate to a sensing or stimulation electrode carried by the lead. In some embodiments, the composite MCRD may also be located proximate to an active fixation element or a passive fixation element.

Implantation of a lead in a body of a patient may cause inflammation of tissue proximate to the implantation location of the lead. In some examples, inflammation of tissue proximate to an electrode carried by the lead may cause variation in stimulation thresholds or sensing thresholds. For example, inflammation can result in unpredictable or excessive stimulation thresholds, which may reduce an effectiveness of stimulation therapy to the patient.

To reduce or minimize inflammation of tissue proximate to the electrode, a lead may include an MCRD, which is located proximate to the electrode and carries a steroid. After implantation of the lead, the steroid may elute from the MCRD to the tissue proximate to the electrode, which may reduce or substantially eliminate inflammation of the tissue. By reducing inflammation of the tissue proximate the electrode, the steroid may facilitate consistent and low stimulation thresholds.

In addition to causing inflammation of tissue, implantation of the lead in the body of the patient also may present a risk of infection. Although infections are relatively rare, reducing occurrence and severity of infections is desirable. One method for reducing or substantially eliminating risk of infection is prophylactically treating the patient with an antimicrobial, such as an antibiotic.

An MCRD may provide an advantageous device for releasing the antimicrobial. An MCRD that releases both a steroid and an antimicrobial may be termed a composite MCRD. A composite MCRD may include a polymer, a steroid mixed in the polymer, and an antimicrobial mixed in the polymer. In some examples, the composite MCRD may include two materials. The first material may include a first polymer and the steroid, and the second material may include a second polymer and the antimicrobial. In some examples, the first material may comprise a first layer including the first polymer and the steroid, and the second material may comprise a second layer including the second polymer and the antimicrobial. The second layer may be formed on the first layer, the first layer may be formed on the second layer, or the first and second layers may be formed adjacent to each other.

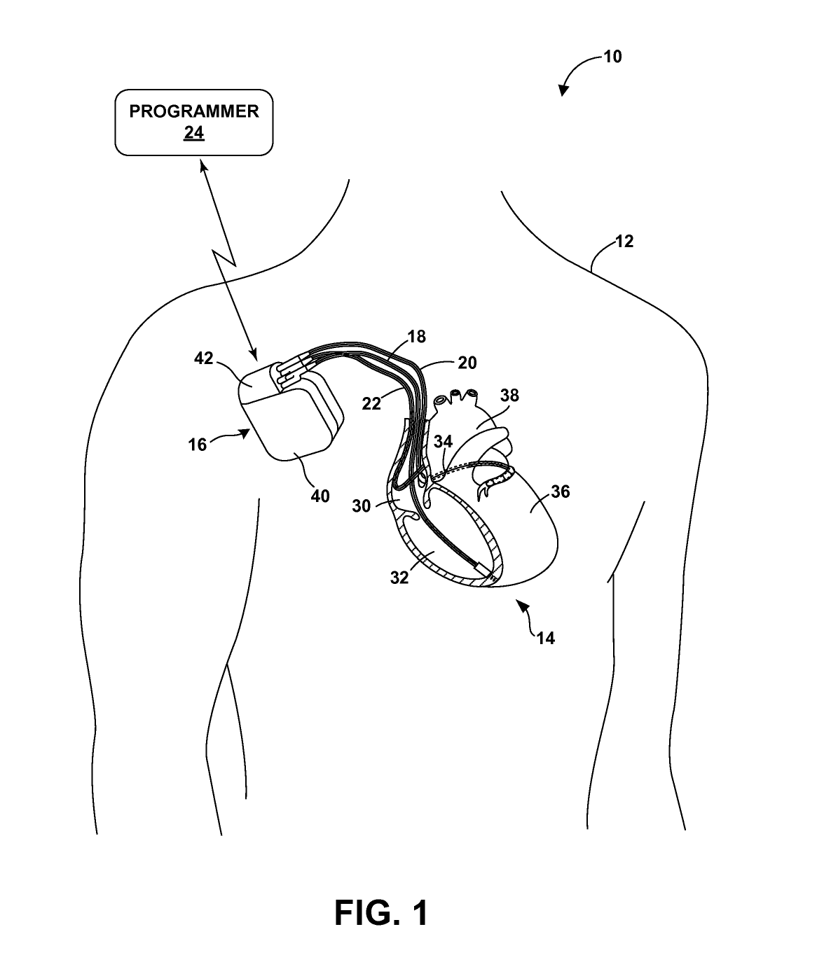

FIG. 1 is a conceptual diagram illustrating an example therapy system 10 that may be used to provide therapy to a patient 12 and which may include a lead comprising a composite MCRD according to one aspect of the disclosure. Patient 12 ordinarily, but not necessarily, will be a human. Therapy system 10 may include an implantable cardiac device (ICD) 16 and a programmer 24. In the example illustrated in FIG. 1, ICD 16 is connected (or “coupled”) to leads 18, 20, and 22. ICD 16 may be, for example, a device that provides cardiac rhythm management therapy to heart 14, and may include, for example, an implantable pacemaker, cardioverter, and/or defibrillator that provides therapy to heart 14 of patient 12 via electrodes coupled to one or more of leads 18, 20, and 22. In some examples, ICD 16 may deliver pacing pulses, but not cardioversion or defibrillation shocks, while in other examples, ICD 16 may deliver cardioversion or defibrillation shocks, but not pacing pulses. In addition, in further examples, ICD 16 may deliver pacing pulses, cardioversion shocks, and defibrillation shocks.

While the example in FIG. 1 is directed to leads 18, 20, and 22 attached to an ICD 16, in other examples, a lead including a composite MCRD may be utilized with other implantable medical devices. For example, a lead according to the disclosure may be attached to an implantable monitoring device that monitors one or more physiological parameter of patient 12, an implantable neurostimulator, such as, for example, a spinal cord stimulator, a deep brain stimulator, a pelvic floor stimulator, or a peripheral nerve stimulator, or the like.

Leads 18, 20, and 22 that are coupled to ICD 16 extend into the heart 14 of patient 12 to sense electrical activity of heart 14 and/or deliver electrical stimulation to heart 14. In the example shown in FIG. 1, right ventricular (RV) lead 18 extends through one or more veins (not shown), the superior vena cava (not shown), right atrium 30, and into right ventricle 32. Left ventricular (LV) coronary sinus lead 20 extends through one or more veins, the vena cava, right atrium 30, and into the coronary sinus 34 to a region adjacent to the free wall of left ventricle 36 of heart 14. Right atrial (RA) lead 22 extends through one or more veins, the vena cava, and into the right atrium 30 of heart 14. In other examples, ICD 16 may deliver stimulation therapy to heart 14 by delivering stimulation to an extravascular tissue site in addition to or instead of delivering stimulation via electrodes of leads 18, 20, and 22.

Leads 18, 20, and 22 (collectively “leads 18”) may be electrically coupled to a signal generator, a sensing module, or another module of ICD 16 via connector block 42. In some examples, proximal ends of leads 18 may include electrical contacts that electrically couple to respective electrical contacts within connector block 42. In addition, in some examples, leads 18 may be mechanically coupled to connector block 42 with the aid of set screws, connection pins or another suitable mechanical coupling mechanism.

Each of the leads 18 includes an elongated insulative lead body, which may carry a number of coiled conductors separated from one another by tubular insulative sheaths. Other lead configurations are also contemplated, such as lead configurations that do not include coiled conductors. In the illustrated example, bipolar electrodes 50 and 52 are located proximate to a distal end of lead 18. In addition, bipolar electrodes 54 and 56 are located proximate to a distal end of lead 20 and bipolar electrodes 58 and 60 are located proximate to a distal end of lead 22.

Electrodes 50, 54, and 58 may take the form of ring electrodes, and electrodes 52, 56, and 60 may take the form of extendable helix tip electrodes mounted retractably within insulative electrode heads 62, 64, and 66, respectively. Electrodes 52, 56, and 60 also may function as active fixation elements to fix leads 18, 20, and 22, respectively, to cardiac tissue. Each of the electrodes 50, 52, 54, 56, 58, and 60 may be electrically coupled to a respective one of the conductors within the lead body of its associated lead 18, 20, and 22, and thereby coupled to respective ones of the electrical contacts on the proximal end of leads 18, 20 and 22.

Electrodes 50, 52, 54, 56, 58, and 60 may sense electrical signals attendant to the depolarization and repolarization of heart 14. The electrical signals are conducted to ICD 16 via conductors within the respective leads 18, 20, and 22. In some examples, ICD 16 also delivers pacing pulses via electrodes 50, 52, 54, 56, 58, and 60 to cause depolarization of cardiac tissue of heart 14. In some examples, as illustrated in FIG. 2, ICD 16 includes one or more housing electrodes, such as housing electrode 68, which may be formed integrally with an outer surface of hermetically-sealed outer housing 40 of ICD 16 or otherwise coupled to housing 40. In some examples, housing electrode 68 is defined by an uninsulated portion of an outward facing (e.g., facing tissue of patient 12 when ICD 16 is implanted within patient 12) portion of housing 40 of ICD 16. In some examples, housing electrode 68 comprises substantially all of housing 40. Electrically insulative divisions between insulated and uninsulated portions of housing 40 may be employed to define two or more housing electrodes. Any of the electrodes 50, 52, 54, 56, 58, and 60 may be used for unipolar sensing or pacing in combination with housing electrode 68. Housing 40 may enclose a signal generator that generates cardiac pacing pulses and defibrillation or cardioversion shocks, as well as a sensing module for monitoring the heart rhythm of patient 12.

Leads 18, 20, and 22 also include elongated electrodes 72, 74, 76, respectively, which may take the form of an exposed coil. ICD 16 may deliver defibrillation pulses to heart 14 via any combination of elongated electrodes 72, 74, and 76, and housing electrode 68. Electrodes 68, 72, 74, and 76 may also be used to deliver cardioversion pulses to heart 14. Electrodes 72, 74, and 76 may be fabricated from any suitable electrically conductive material, including, but not limited to, platinum, a platinum alloy or other materials known to be usable in implantable defibrillation electrodes.

Although not illustrated in FIGS. 1 and 2, leads 18 may each include a composite MCRD which elutes a steroid and an antimicrobial after implantation of leads 18 in heart 14. A composite MCRD may be located proximate to at least one of electrodes 50, 52, or 72 carried by lead 18, at least one of electrodes 54, 56, or 74 carried by lead 20, and/or at least one of electrodes 58, 60, or 76 carried by lead 22. In some examples, a composite MCRD may be located proximate to electrode 52, which also acts as a fixation element for attaching lead 20 to tissue of right ventricle 32. For example, a composite MCRD may be coupled to insulative electrode head 62, as will be described below. Similarly, a composite MCRD may be located proximate to electrode 56 and/or electrode 60. Inclusion of a composite MCRD proximate to a fixation element may be advantageous as a site of tissue injury (e.g., where an active fixation element is introduced into tissue) may be susceptible to infection.

The composite MCRD may include a polymer, a steroid, and an antimicrobial. In some examples, the composite MCRD may include two materials. The first material may include a first polymer and the steroid, while the second material may include a second polymer and the antimicrobial. The first polymer and the second polymer may be the same or may be different. In some examples, the first material may comprise a first layer including the first polymer and the steroid, and the second material may comprise a second layer including the second polymer and the antimicrobial. The second layer may be formed on the first layer, the first layer may be formed on the second layer, or the first and second layers may be formed adjacent to each other.

The polymer may be a biocompatible polymer. For example, the polymer may include at least one of silicone, polyurethane, collagen, poly(lactic-co-glycolic acid) (PLGA), poly(lactic acid) (PLA), poly(glycolic acid) (PGA), poly(ethylene oxide) (PEO), poly(ortho ester) (POE), poly(ε-caprolactone), poly(dioxanone), polyglyconate, hyaluronic acid, gelatin, fibrin, fibrinogen, cellulose, starch, cellulose acetate, polypyrrolidone (PVP), a poly(ethylene oxide)/poly(propylene oxide) copolymer (PEO-PPO), poly(ethylene vinyl acetate), poly(hydroxybutyrate-covalerate), polyanhydride, poly(glycolic acid-co-trimethylene carbonate), polyphosphoester, polyphosphoester urethane, a poly(amino acid), a cyanoacrylate, poly(trimethylene carbonate), poly(iminocarbonate), a copoly(ether-ester) such as PEO/PLA, a polyalkylene oxalate, a polyphasphazene, a polyarylate, a polyacrylate, poly(vinyl alcohol), poly(vinyl acetate), carboxymethyl cellulose, poly(acrylic acid), a sugar ester, or the like. In some examples, the first and second materials may each comprise silicone or may each comprise collagen. In other examples, the first material may comprise silicone and the second material may comprise collagen. Of course, other combinations of polymers are contemplated and within the scope of the disclosure. For example, a single-material composite MCRD or at least one of the first material or the second material may comprise a mixture of at least two polymers.

In examples in which at the composite MCRD includes collagen, the composite MCRD may include collagen alone or in combination with another biocompatible polymer. The collagen may comprise collagen from an animal (xenogenous collagen) or from a human (autologous or allogenic collagen). The collagen may comprise at least one collagen type, such as Type-I, -II, -III, -IV, -VII, or -IX. Collagen Type-I may be obtained from animal tissue such as skin, tendons, or the like. In some examples, the collagen may be enzymatically treated prior to use, while in other examples, the collagen may not be enzymatically treated prior to use.

The composite MCRD also includes a steroid. In some examples, the steroid may include beclamethasone or dexamethasone (DXM), a glucocorticoid. The DXM may be incorporated into the composite MCRD as DXM, or may be incorporated in the MCRD as a pro-drug, such as dexamethsone sodium phosphate (DSP) or dexamethasone acetate (DXAC). A pro-drug is a pharmacologically inactive compound that is designed in increase an amount of the active species (the drug) that reaches the site of action (e.g., a tissue site proximate to the implant location). After implantation, pro-drugs are converted to biologically active metabolites. For example, DSP and DXAC are hydrolyzed to DXM, which is the active species. In the case of DXM, the three forms in which it may be provided (DXM, DSP, and DXAC) may provide flexibility in designing and implementing composite MCRDs. For example, DXM, DSP, and DXAC may have different solubilities in a polymer or different hydrophilicities or hydrophobicities, which may affect an elution rate of the steroid from the MCRD. As another example, DXM, DSP, and DXAC may have different compatibilities with the antimicrobial included in the MCRD.

The composite MCRD further includes an antimicrobial. The antimicrobial may include, for example, an antibiotic such as tetracyclines (e.g. minocycline), rifamycins (e.g. rifampin), macrolides (e.g. erythromycin), penicillins (e.g. nafcillin), cephalosporins (e.g. cefazolin), other beta-lactam antibiotics (e.g. imipenem, aztreonam) aminoglycosides (e.g. gentaminicn), glycopeptides (e.g. vancomycin), quinolones (e.g. ciprofloxacin), fusidic acid, trimethoprim, metronidazole, mupirocin, polenes (e.g. amphotericin B), azoles (e.g. fluconazole) and beta-lactam inhibitors (e.g. sulbactam), tigecycline, daptomycin, clindamycin, or another fluoroquinolone, an antiseptic, an antimicrobial peptide, a quaternary ammonium, or the like. In some examples, the antimicrobial may be provided in a salt form, e.g., gentamicin crobefate or gentamicin sulfate. In some examples, at least two antimicrobials may be selected to efficaciously prevent or treat any infection present proximate to the implant location of leads 18, 20, 22. For example, gentamicin may be utilized alone or in combination with at least one other antimicrobial.

The composite MCRD may be formed via, for example, injection molding, compression molding, transfer molding, casting, solvent dispersion followed by casting, spraying, extruding, painting, or the like. In examples in which the composite MCRD includes two materials, the second material may be molded to the first material, pressed to the first material, solvent fused to the first material, coated on the first material or the like.

In some examples, the steroid and the antimicrobial may be mixed into the polymer before forming the polymer into the form factor of the composite MCRD. For example, the steroid and/or the antimicrobial may be mixed in one or both constituent of a two part silicone prior to mixing the two parts together and curing the silicone. As another example, the polymer, the steroid, and the antimicrobial may be mixed in one or more solvent, homogenized, and the solvent may be removed during solvent casting to produce the composite MCRD.

In other examples, the steroid and/or the antimicrobial may be deposited into the composite MCRD after forming the polymer into the desired form factor. For example, the steroid and/or antimicrobial may be mixed into a solvent and the polymer may be coated or impregnated with the steroid and/or antimicrobial by submerging, spraying, washing, or coating the polymer with the steroid and/or antimicrobial solution. As another example, the steroid and/or antimicrobial may be mixed into a solvent and the steroid and/or antimicrobial may be deposited into pores of a porous polymer layer by submerging spraying, washing, or coating the porous polymer with the solution and then drying the porous polymer layer to remove the solvent and leave the steroid and/or antimicrobial. In some examples, the steroid and the antimicrobial may be deposited in the polymer in separate steps or by different methods.

In some examples in which the composite MCRD includes two materials, the steroid may be deposited in the first material and the antimicrobial may be deposited in the second material prior to coupling the first material and the second material. Any suitable technique may be utilized to deposit the steroid in the first material and the antimicrobial in the second material. In some examples, the steroid may be deposited in the first material using a different technique that the technique used to deposit the antimicrobial in the second material. In other examples, the same technique may be utilized to deposit the steroid in the first material and the antimicrobial in the second material.

Additional details regarding formation of a polymer material or layer including a polymer and an antimicrobial, which may be adapted to forming a polymer material or layer including a polymer and a steroid or a polymer, a steroid, and an antimicrobial may be found in U.S. Provisional Patent Application Ser. No. 61/149,214, filed on Feb. 2, 2009; U.S. Provisional Patent Application Ser. No. 61/152,467, filed on Feb. 13, 2009; U.S. Provisional Patent Application Ser. No. 61/165,273, filed on Mar. 31, 2009; U.S. Provisional Patent Application Ser. No. 61/186,279, filed on Jun. 11, 2009; U.S. Provisional Patent Application Ser. No. 61/218,328, filed on Jun. 18, 2009; and U.S. Provisional Patent Application Ser. No. 61/256,758, filed on Oct. 30, 2009. The entire disclosure of each of these applications is incorporated herein by reference.

Returning to FIG. 1, in some examples, programmer 24 may be a handheld computing device or a computer workstation. Programmer 24 may include a user interface that receives input from a user. The user interface may include, for example, a keypad and a display, which may be, for example, a cathode ray tube (CRT) display, a liquid crystal display (LCD) or light emitting diode (LED) display. The keypad may take the form of an alphanumeric keypad or a reduced set of keys associated with particular functions. Programmer 24 can additionally or alternatively include a peripheral pointing device, such as a mouse, via which a user may interact with the user interface. In some embodiments, a display of programmer 24 may include a touch screen display, and a user may interact with programmer 24 via the display.

A user, such as a physician, technician, or other clinician, may interact with programmer 24 to communicate with ICD 16. For example, the user may interact with programmer 24 to retrieve physiological or diagnostic information from ICD 16. A user may also interact with programmer 24 to program ICD 16, e.g., select values for operational parameters of ICD 16.

Programmer 24 may communicate with ICD 16 via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the ICD 16 implant site in order to improve the quality or security of communication between ICD 16 and programmer 24.

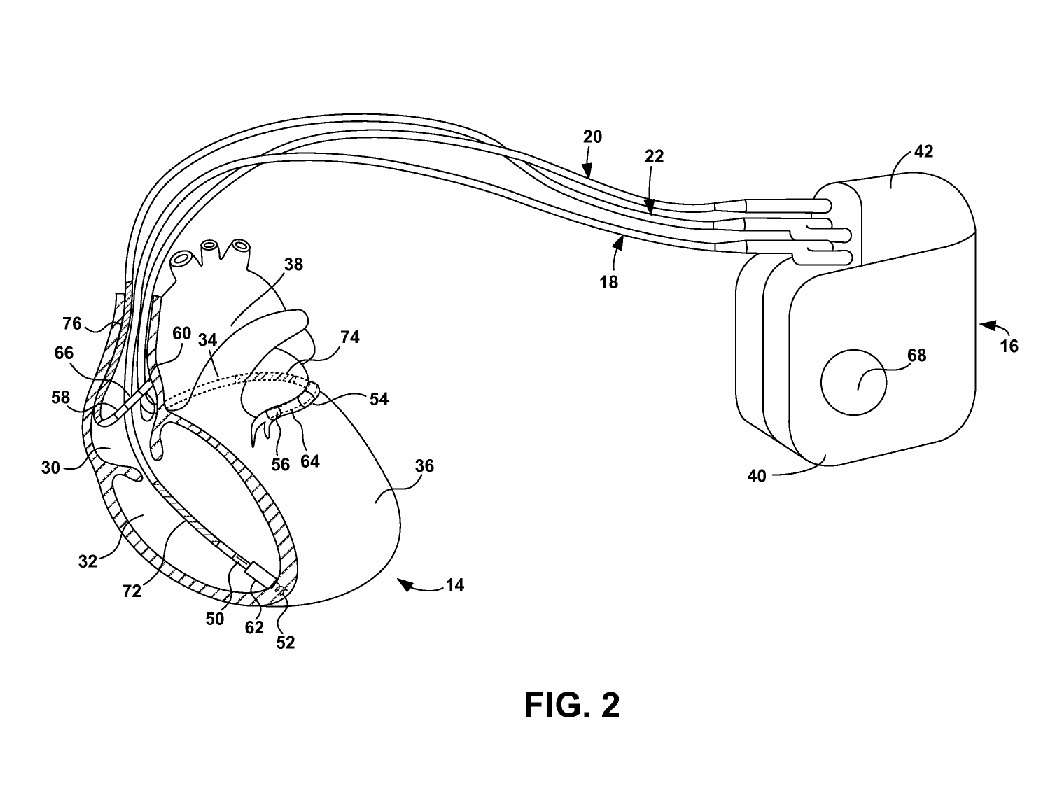

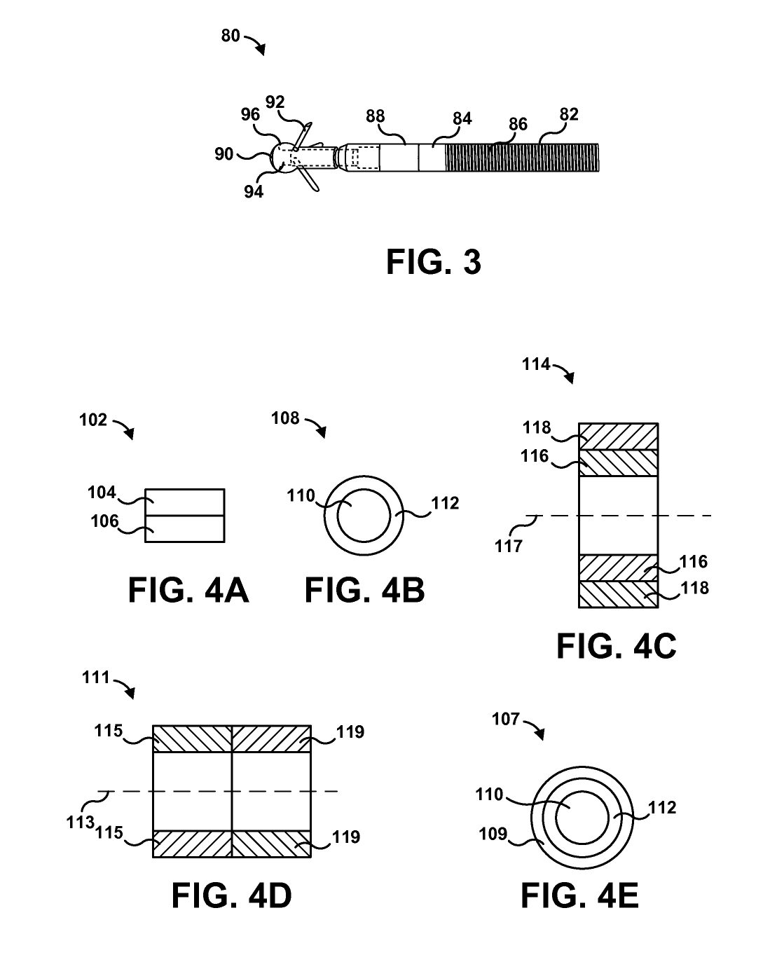

FIG. 3 illustrates a perspective diagram of an example of a distal end of a lead that includes a composite MCRD according to the present disclosure. Lead 80 includes a lead body 82, the distal end of which is shown in FIG. 3. Lead body 82 includes an outer insulative sheath 84, which encloses at least one coiled conductor 86. Outer insulative sheath 84 may comprise, for example, silicone, polyurethane, or another biocompatible polymer.

Lead 80 further includes a first electrode 88 formed proximate to the distal end of the lead 80 and a second electrode 90, also formed proximate to the distal end. First electrode 88 may be a ring electrode, as shown in FIG. 3. Second electrode 90 comprises a tip electrode. In other examples, one or both of first electrode 88 and second electrode 90 may be a paddle electrode, a segmented ring electrode (e.g., multiple distinct electrodes formed at different circumferential portions at a single axial position of lead 80), a helical tip electrode, or another type of electrode. In general, lead 80 may include any type of electrode, and is not limited to those electrodes described herein.

First and second electrode 88, 90 may comprise biocompatible, conductive metal or alloy. For example, first electrode 88 and/or second electrode 90 may comprise platinum, a platinum alloy (e.g., Pt/Ir), titanium, a titanium alloy, or the like. In some examples, at least one of first electrode 88 or second electrode 90 may include a porous material, such as sintered titanium or sintered platinum.

Lead 80 also includes a passive fixation element 92. Passive fixation element 92 comprises a plurality of tines, which extend in a radial direction from lead body 82. As shown in FIG. 3, passive fixation element 92 is located proximate to first electrode 88 and second electrode 90. In some examples, lead 80 may include more than one passive fixation element 92. Although passive fixation element 92 is illustrated as a plurality of tines, in other examples, fixation element 92 may comprise, for example, an anchor skirt, a hydrogen disk, or the like. Other passive fixation elements are contemplated and fall within the scope of the disclosure.

Passive fixation element 92 may comprise, for example, a polymer, such as silicone, polyurethane, or the like. In some examples, passive fixation element 92 may include a hydrogel, which swells when exposed to bodily fluids and may expand in at least one dimension. In still other examples, passive fixation element 92 may comprise a resilient, biocompatible metal. Passive fixation element 92 may be configured to flatten against lead body 82 when being implanted into patient 12 and to extend once the lead 80 has been implanted. For example, lead body 82 may be implanted through a cannula or other introducer, which comprises a cylindrical body defining a lumen through which lead 80 is implanted. Passive fixation element 92 may be forced against lead body 82 while lead 80 is introduced through the cannula, a passive fixation element 92 may resiliently expand from lead body 82 when the cannula is withdrawn.

Lead 80 also includes a composite MCRD 94, which is located within lead body 82 proximate to first electrode 88 and second electrode 90. In the example illustrated in FIG. 3, composite MCRD 94 is located within electrode tip 96 of lead 80. Composite MCRD 94 may include a biocompatible polymer, a steroid, and an antimicrobial. The steroid and the antimicrobial may elute from composite MCRD 94, through pores of second electrode 90, and to tissue proximate lead 80.

The biocompatible polymer in composite MCRD 94 may be biodegradable or non-biodegradable. For example, the biocompatible polymer may include at least one of silicone, polyurethane, collagen, PLGA, PLA, PGA, PEO, POE, poly(ε-caprolactone), poly(dioxanone), polyglyconate, hyaluronic acid, gelatin, fibrin, fibrinogen, cellulose, starch, cellulose acetate, PVP, a PEO-PPO copolymer, poly(ethylene vinyl acetate), poly(hydroxybutyrate-covalerate), polyanhydride, poly(glycolic acid-co-trimethylene carbonate), polyphosphoester, polyphosphoester urethane, a poly(amino acid), a cyanoacrylate, poly(trimethylene carbonate), poly(iminocarbonate), a copoly(ether-ester) such as PEO/PLA, a polyalkylene oxalate, a polyphasphazene, a polyarylate, a polyacrylate, poly(vinyl alcohol), poly(vinyl acetate), carboxymethyl cellulose, poly(acrylic acid), a sugar ester, or the like. One example of a silicone that may be used in composite MCRD 94 is available under the trade designation Silastic® Q-4765, from Dow Corning Corp., Midland, Mich. In some examples, composite MCRD 94 may include a mixture of two or more biocompatible polymers. Use of a biodegradable polymer may facilitate release of substantially all of the steroid and antimicrobial in composite MCRD 94.

In some examples, composite MCRD 94 may comprise collagen, alone or in combination with at least one other polymer. The collagen may comprise collagen from an animal (xenogenous collagen) or from a human (autologous or allogenic collagen). The collagen may comprise at least one collagen type, such as Type-I, -II, -III, -IV, -VII, or -IX. Collagen Type-I may be obtained from animal tissue such as skin, tendons, or the like. In some examples, the collagen may be enzymatically treated prior to use, while in other examples, the collagen may not be enzymatically treated prior to use.

The antimicrobial in composite MCRD 94 may include, for example, an antibiotic such as minocycline, clindamycin, rifampin, tigecycline, daptomycin, gentamicin, or another fluoroquinolone, an antiseptic, an antimicrobial peptide, a quaternary ammonium, or the like. In some examples, the antimicrobial may be provided in a salt form, e.g., gentamicin crobefate or gentamicin sulfate. In some examples, two or more antimicrobials may be selected to efficaciously prevent or treat any infection present proximate to the implant location of lead 80. For example, gentamicin may be utilized alone or in combination with at least one other antimicrobial.

The steroid in composite MCRD 94 may include, for example, beclamethasone or DXM, a glucocorticoid. The DXM may be incorporated into the composite MCRD as DXM, or may be incorporated in the MCRD as a pro-steroid, such as DSP or DXAC. A pro-steroid is a pharmacologically inactive compound that is designed in increase an amount of the active species (the drug) that reaches the site of action (e.g., a tissue site proximate to the implant location). After implantation, a pro-steroid are converted to a biologically active steroid. For example, DSP and DXAC are hydrolyzed to DXM, which is the active species. In the case of DXM, the three forms in which it may be provided may provide flexibility in designing and implementing composite MCRDs. For example, DXM, DSP, and DXAC may have different solubilities in a polymer, which may affect an elution rate of the steroid from the MCRD. As another example, DXM, DSP, and DXAC may have different compatibilities with the antimicrobial included in the MCRD. In some examples, composite MCRD 94 may include a steroid other than DXM, DSP, or DXAC.

In some examples, composite MCRD 94 may include two materials. In some examples, the first material may be formed as a first layer and the second material may be formed as a second layer. A first material may include the steroid and a first polymer and a second material may include the antimicrobial and a second polymer. The first polymer and the second polymer may be the same or may be different. The first material and second material may be arranged in various configurations, as illustrated in FIGS. 4A-4E. For example, as shown in FIG. 4A, a composite MCRD 102 may include a second material 104 formed as at least one layer on a first material 106. First material 106 may be formed as at least one layer (e.g., first material 106 may comprise multiple, substantially homogeneous layers that combine to form the thickness of first material 106). Composite MCRD 102 may be shaped as a cylinder, a rectangular polygon, or another three-dimensional solid. For example, second material 104 may comprise a cylinder formed on a planar surface of a cylindrical first material 102.

As another example, FIG. 4B illustrates an end view of a composite MCRD 108 including a cylindrical first material 110 and a second material 112 formed as an annulus around cylindrical first material 110. In some examples, second material 112 may be cylindrical and first material 110 may be an annulus around second material 112. In other words, either the cylindrical layer or the annular layer may include the steroid, and the other layer may include the antimicrobial.

FIG. 4C illustrates an additional example in which a composite MCRD 114 includes a first material formed as a first annular layer 116 and a second material formed as a second annular layer 118. In the example of FIG. 4C, first annular layer 116 is located radially inward from second annular layer 118 (relative to longitudinal axis 117). In other examples, second annular layer 118 may be located radially inward from first annular layer 116. Additionally, as shown in FIG. 4D, in some embodiments, a composite MCRD 111 may include a first material formed as a first annular layer 115 and a second material formed as a second annular layer 119, and the layers 115, 119 may be located substantially equidistant from longitudinal axis 113 in a radial direction, but at different positions along longitudinal axis 113.

While the examples illustrated in FIGS. 4A-4D illustrate a composite MCRD that includes a first material including a polymer and a steroid and a second material including a polymer and an antimicrobial, in some examples, a composite MCRD may include more than two materials. For example, as shown in FIG. 4E, a composite MCRD 107 may include a first material 110 that includes a first polymer and a steroid, a second material 112 that includes a second polymer and a first antimicrobial, and a third material 109 that includes a third polymer and a second antimicrobial. In some examples, the first, second, and third polymers may be the same, while in other examples, at least one of the first, second, and third polymers may be different than another of the first, second, and third polymers. Each of the first, second, and third polymers may comprise a biocompatible polymer, such as, for example, a biocompatible polymer listed herein.

In some examples, the first antimicrobial may be the same as the second antimicrobial. In examples in which the first antimicrobial and the second antimicrobial are the same, second material 112 and third material 109 may be utilized to influence release characteristics, such as an elution rate or elution duration, of the antimicrobial. For example, third material 109 may comprise a polymer which is formed to be more porous that the polymer in second material 112. In this way, the antimicrobial may elute more quickly from third material 109 to provide “burst” elution of the antimicrobial, while the antimicrobial may elute more slowly from second material 112, which may provide more extended delivery of the antimicrobial.

Similarly, second material 112 may comprise a first polymer that is different from a second polymer from which third material 109 is formed. The first and second polymers may be selected to affect the rate at which the antimicrobial is eluted from composite MCRD 107. For example, second material 112 may be formed from a polymer in which the antimicrobial is more soluble, and third material 109 may be formed from a polymer in which the antimicrobial is less soluble. In this way, the antimicrobial may elute more quickly from third material 109 than from second material 112.

In other examples, the first antimicrobial may be different than the second antimicrobial. The first and second antimicrobials may be selected to efficaciously prevent or treat any infection present proximate to the implant location of the lead to which composite MCRD 107 is attached. In some examples, the first and second antimicrobials may comprise different salt forms of a similar active agent, such as gentamicin sulfate and gentamicin crobefate. In some examples, one of the salt forms may be more hydrophobic and the other salt form may be more hydrophilic. In such examples, the relative elution rates of the salt forms may be different, and may allow control of an elution profile of the antimicrobial from the composite MCRD 107.

Although not shown in FIGS. 4A-4E, in some examples, a composite MCRD may include three or more materials formed in layers located substantially equidistant from a longitudinal axis of a lead (e.g., longitudinal axis 113, shown in FIG. 4D) in a radial direction, but at different positions along the longitudinal axis.

Regardless of the configuration of the first material 104, 110, 112 and the second material 106, 112, 118 (hereafter “first material 104” and “second material 106”), in some examples first material 104 and second material 106 may comprise the same polymer or mixture of polymers, while in other examples, first material 104 may comprise a different polymer or mixture of polymers than second material 106. For example, first material 104 may include silicone and/or polyurethane and the steroid, and second material 106 may include collagen and the antimicrobial. As another example, first material 104 may comprise collagen and the steroid and second material 106 may comprise collagen and the antimicrobial. Other examples are contemplated and are within the scope of the disclosure.

In some examples, whether formed as a single material or two materials, composite MCRD 94 may include other components that may influence the properties of the MCRD 94. For example, composite MCRD 94 may include an antioxidant mixed in the polymer and/or the antimicrobial, which may reduce or substantially prevent oxidation of the antimicrobial. Exemplary antioxidants include, but are not limited to, monofunctional hindered phenolic antioxidants, such as those available under the trade designations Irganox 1076 and Irganox 1010 from Ciba Corp., Tarrytown, N.Y., butylated hydroxyl toluene (BHT), vitamin E, vitamin A, or vitamin C. In some examples, composite MCRD 94 or a material 104, 106 of composite MCRD 94 may include between approximately 0.1 wt. % and approximately 2 wt. % antioxidant.

In some examples, composite MCRD 94 may additionally or alternatively include a wetting agent, such as a salt, which facilitates wetting of MCRD 94 and elution of the steroid and the antimicrobial from the MCRD 94. In some examples, one or more of the steroid, the antimicrobial, or the polymer may function as a wetting agent. For example, DSP is a wetting agent, and an antimicrobial salt may be a wetting agent.

While not shown in FIG. 3, in some examples, lead 80 may include a second composite MCRD located proximate to first electrode 88. For example, the second composite MCRD may be disposed within lead body 82 underneath first electrode 88, which may comprise a porous metal or alloy. The antimicrobial and steroid in second composite MCRD may then elute from the second composite MCRD, through pores in first electrode 88 and to tissue of patient 12 proximate to first electrode 88. Other configurations of a second composite MCRD are also contemplated. For example, a second MCRD may be formed as an annulus about a portion of first electrode 88.

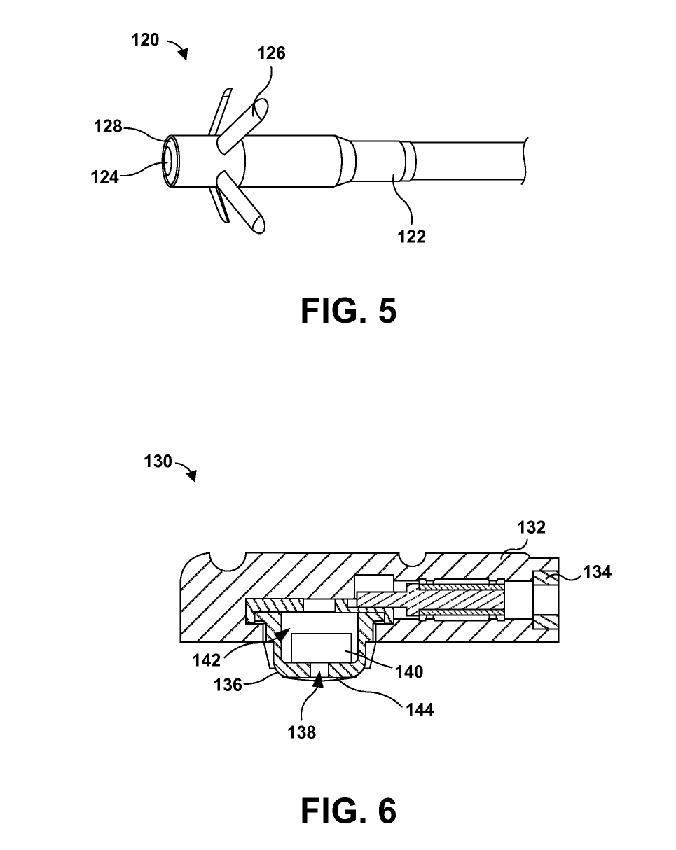

FIG. 5 illustrates another example of a distal end of a lead 120 including an electrode 124, a passive fixation element 126, and a composite MCRD 128. Electrode 124 is a tip electrode, and is located at the distal tip of lead 120. Composite MCRD 128 is also located at the distal tip of lead 120, proximate to and partially surrounding electrode 124. Lead 120 also includes a passive fixation element 126 coupled to lead body 122 proximate to electrode 124 and composite MCRD 128.

Similar to composite MCRD 94, composite MCRD 128 may comprise a biocompatible polymer, a steroid, and an antibiotic. In some examples, composite MCRD 128 may comprise a single material, and the steroid and the antimicrobial may be mixed in the single material. In other examples, composite MCRD 128 may comprise two materials, similar to composite MCRD 114 illustrated in FIG. 4C. In some examples, composite MCRD 128 may comprise silicone, DXAC, and an antimicrobial. Silicone and DXAC may be utilized because composite MCRD 128 then may be dimensionally stable (i.e., may not significantly change in size) when wetted by bodily fluids upon implantation of lead 120 in patient 12. In other examples, composite MCRD 128 may comprise a combination of any other polymers, steroids, or antimicrobials described herein.

FIG. 6 illustrates a distal end of a lead 130 that does not include a passive fixation element, but which includes a composite MCRD 140. Lead 130 may be an epicardial lead, which is implanted proximate to epicardial tissue of heart 14. In some examples, lead 130 may be fixed in position by a suture or another fixation element. Electrode 136 protrudes from a surface of lead body 132 and is connected to a conductor 134 within lead body 132 of lead 130.

Composite MCRD 140 is located within a cavity 142 formed in an interior of electrode 136. Similar to electrode 90 of lead 80 shown in FIG. 3, electrode 136 may comprise a porous material, such as sintered titanium or sintered platinum. In other examples, electrode 136 may include a nonporous conductive material, such as a biocompatible metal or metal alloy.

Electrode 136 may optionally include a channel 138 extending from cavity 142 to an exterior of electrode 136. Channel 138 may facilitate wetting of composite MCRD 140 with bodily fluid after implantation lead 130 and elution of the steroid and antimicrobial from the MCRD 140.

Electrode 136 may also optionally include a coating 144 of steroid and/or antimicrobial, which may provide an initial burst elution of steroid and/or antimicrobial after implantation of lead 130 in patient 12.

As described above, composite MCRD 140 may include a single material comprising a polymer, the steroid, and the antimicrobial, or may include a first material comprising a polymer and the steroid and a second material comprising a polymer and the antimicrobial. For example, composite MCRD 140 may comprise a first material and a second material similar to composite MCRD 102 illustrated in FIG. 4A.

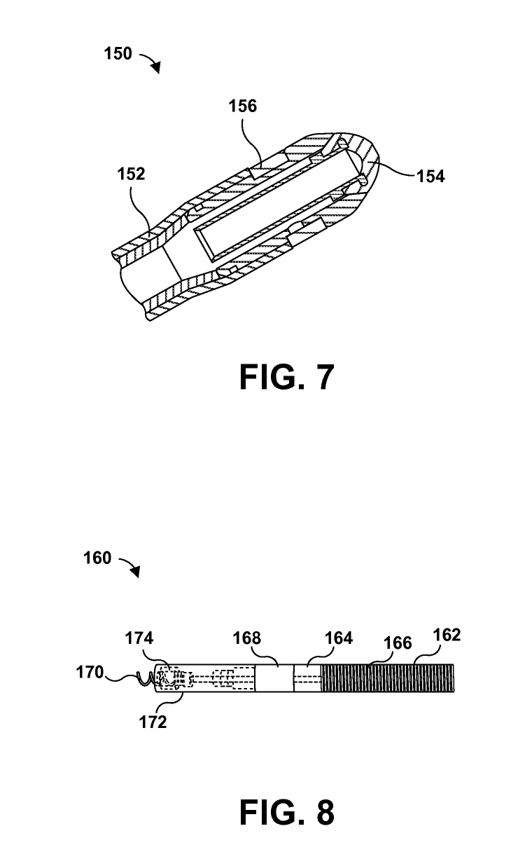

FIG. 7 is a cross-sectional diagram illustrating a distal end of another example of a lead 150 including a composite MCRD 156. Similar to FIG. 6, lead 150 does not include a fixation element coupled to lead body 152. In some examples, lead body 152 may be sutured to tissue in patient 12 to maintain a position of lead 150 relative to an initial implantation location. In other examples, a position of lead 150 may be maintained by geometry or another physical characteristic of lead 150. For example, lead body 152 may be curved to position tip electrode 154 adjacent to tissue of heart 14 and maintain contact between the tissue of heart 14 and tip electrode 154.

Lead 150 includes a composite MCRD 156 formed as an annulus about lead body 152, proximate to tip electrode 154. In some examples, composite MCRD 156 includes a single material including a polymer, a steroid, and an antimicrobial. In other examples, composite MCRD 156 includes a first material including a first polymer and a steroid and a second material including a second polymer and a steroid. In some examples, MCRD 156 may include first and second materials configured as illustrated in FIG. 4C, while in other examples, the first material and the second material may be positioned next to each other along a longitudinal axis of lead 150.

FIG. 8 is a conceptual diagram of a distal end of an example of a lead including a composite MCRD proximate to an active fixation element. Lead 160 includes a lead body 162, the distal end of which is shown in FIG. 8. Lead body 162 includes an outer insulative sheath 164, which encloses at least one coiled conductor 166. Outer insulative sheath 164 may comprise, for example, silicone, polyurethane, or another biocompatible polymer.

Lead 160 further includes a first electrode 168 formed proximate to the distal end of the lead 160 and a second electrode 170, also formed proximate to the distal end. First electrode 168 may be a ring electrode, as shown in FIG. 8. Second electrode 90 comprises a retractable helical tip electrode. In other examples, one or both of first electrode 168 and second electrode 170 may be a paddle electrode, a segmented ring electrode (e.g., multiple distinct electrodes formed at different circumferential portions at a single axial position of lead 160), a helical tip electrode, or another type of electrode. In general, lead 160 may include any type of electrode, and is not limited to those electrodes described herein.

First and second electrode 168, 170 may comprise a biocompatible, conductive metal or alloy. For example, first electrode 168 and/or second electrode 170 may comprise platinum, a platinum alloy (e.g., Pt/Ir), titanium, a titanium alloy, or the like. In some examples, at least one of first electrode 168 or second electrode 170 may include a porous material, such as sintered titanium or sintered platinum.

In addition to being an electrode, second electrode 170 functions as an active fixation element. As lead 160 is implanted in the body of patient 12 (e.g., in heart 14), second electrode 170 may be retracted into sheath 172 so that second electrode 170 does not protrude from the distal tip of lead 160. Once lead 160 has been advanced within heart 14 to the desired position, second electrode 170 may be extended while being rotated to screw the electrode into tissue of heart 14 and fix lead 160 to the tissue.

Lead 160 also includes a composite MCRD 174 disposed within sheath 172 proximate to second electrode 170. Composite MCRD 94 may include a biocompatible polymer, a steroid, and an antimicrobial. The steroid and the antimicrobial may elute from composite MCRD 94 to tissue proximate lead 80 through the lumen defined by sheath 172.

As described above, the biocompatible polymer in composite MCRD 174 may be biodegradable or non-biodegradable. For example, the biocompatible polymer may include at least one of silicone, polyurethane, collagen, PLGA, PLA, PGA, PEO, POE, poly(ε-caprolactone), poly(dioxanone), polyglyconate, hyaluronic acid, gelatin, fibrin, fibrinogen, cellulose, starch, cellulose acetate, PVP, a PEO-PPO copolymer, poly(ethylene vinyl acetate), poly(hydroxybutyrate-covalerate), polyanhydride, poly(glycolic acid-co-trimethylene carbonate), polyphosphoester, polyphosphoester urethane, a poly(amino acid), a cyanoacrylate, poly(trimethylene carbonate), poly(iminocarbonate), a copoly(ether-ester) such as PEO/PLA, a polyalkylene oxalate, a polyphasphazene, a polyarylate, a polyacrylate, poly(vinyl alcohol), poly(vinyl acetate), carboxymethyl cellulose, poly(acrylic acid), a sugar ester, or the like. One example of a silicone that may be used in composite MCRD 174 is available under the trade designation Silastic® Q-4765, from Dow Corning Corp., Midland, Mich. In some examples, composite MCRD 174 may include a mixture of two or more biocompatible polymers. Use of a biodegradable polymer may facilitate release of substantially all of the steroid and antimicrobial in composite MCRD 174.

In some examples, composite MCRD 174 may comprise collagen, alone or in combination with at least one other polymer. The collagen may comprise collagen from an animal (xenogenous collagen) or from a human (autologous or allogenic collagen). The collagen may comprise at least one collagen type, such as Type-I, -II, -III, -IV, -VII, or -IX. Collagen Type-I may be obtained from animal tissue such as skin, tendons, or the like. In some examples, the collagen may be enzymatically treated prior to use, while in other examples, the collagen may not be enzymatically treated prior to use.

The antimicrobial in composite MCRD 174 may include, for example, an antibiotic such as minocycline, clindamycin, rifampin, tigecycline, daptomycin, gentamicin, or another fluoroquinolone, an antiseptic, an antimicrobial peptide, a quaternary ammonium, or the like. In some examples, the antimicrobial may be provided in a salt form, e.g., gentamicin crobefate or gentamicin sulfate. In some examples, two or more antimicrobials may be selected to efficaciously prevent or treat any infection present proximate to the implant location of lead 160. For example, gentamicin may be utilized alone or in combination with at least one other antimicrobial.

The steroid in composite MCRD 174 may include, for example, beclamethasone or DXM, a glucocorticoid. The DXM may be incorporated into the composite MCRD as DXM, or may be incorporated in the MCRD as a pro-steroid, such as DSP or DXAC. A pro-steroid is a pharmacologically inactive compound that is designed in increase an amount of the active species (the drug) that reaches the site of action (e.g., a tissue site proximate to the implant location). After implantation, a pro-steroid are converted to a biologically active steroid. For example, DSP and DXAC are hydrolyzed to DXM, which is the active species. In the case of DXM, the three forms in which it may be provided may provide flexibility in designing and implementing composite MCRDs. For example, DXM, DSP, and DXAC may have different solubilities in a polymer, which may affect an elution rate of the steroid from the MCRD. As another example, DXM, DSP, and DXAC may have different compatibilities with the antimicrobial included in the MCRD. In some examples, composite MCRD 174 may include a steroid other than DXM, DSP, or DXAC.

While not shown in FIG. 8, in some examples, lead 160 may include a second composite MCRD located proximate to first electrode 168. For example, the second composite MCRD may be disposed within lead body 162 underneath first electrode 168, which may comprise a porous metal or alloy. The antimicrobial and steroid in second composite MCRD may then elute from the second composite MCRD, through pores in first electrode 168 and to tissue of patient 12 proximate to first electrode 168. Other configurations of a second composite MCRD are also contemplated. For example, a second MCRD may be formed as an annulus about a portion of first electrode 168.

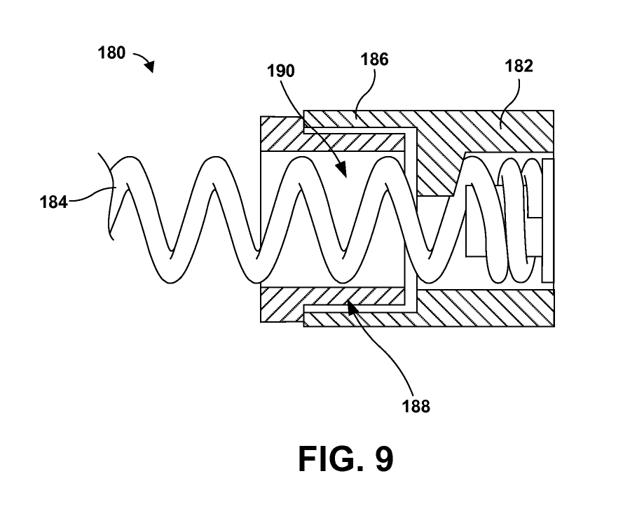

FIG. 9 is a cross-sectional diagram of a distal end of an example of a lead including a composite MCRD proximate to an active fixation element. Distal portion of lead 180 includes a lead body 182 and a sheath 186. As described above with respect to FIG. 8, lead body 182 may comprise silicone, polyurethane, or another biocompatible polymer. Sheath 186 defines a cavity 190 within which retractable helical tip electrode 184 may be retracted. Retractable helical tip electrode 184 is shown extended from cavity 190 in FIG. 8.

Lead 180 further includes a composite MCRD 188 disposed partially within cavity 190 defined by sheath 186. Composite MCRD 188 may comprise a polymer, a steroid, and an antimicrobial. In some examples, composite MCRD 188 may include a first material including a first polymer and a steroid and a second material including a second polymer and an antimicrobial. In some examples, as illustrated in FIG. 4C, composite MCRD 188 may include two concentrically disposed materials, while in other examples, as shown in FIG. 4D, the first and second materials may be disposed adjacent to each other along a longitudinal axis of lead 180.

A composite MCRD (e.g., composite MCRD 94 shown in FIG. 3) may be formed via one of a plurality of techniques. As described above, in some examples, composite MCRD 94 may be formed as a single material including a polymer, a steroid and an antimicrobial. The polymer may include a biocompatible polymer, which may or may not be biodegradable. Examples of polymers that may be used in composite MCRD 94 may include, for example, silicone, polyurethane, collagen, PLGA, PLA, PGA, PEO, POE, poly(ε-caprolactone), poly(dioxanone), polyglyconate, hyaluronic acid, gelatin, fibrin, fibrinogen, cellulose, starch, cellulose acetate, PVP, a PEO-PPO copolymer, poly(ethylene vinyl acetate), poly(hydroxybutyrate-covalerate), polyanhydride, poly(glycolic acid-co-trimethylene carbonate), polyphosphoester, polyphosphoester urethane, a poly(amino acid), a cyanoacrylate, poly(trimethylene carbonate), poly(iminocarbonate), a copoly(ether-ester) such as PEO/PLA, a polyalkylene oxalate, a polyphasphazene, a polyarylate, a polyacrylate, poly(vinyl alcohol), poly(vinyl acetate), carboxymethyl cellulose, poly(acrylic acid), a sugar ester, or the like. Examples of steroids include beclamethasone, DXM, DSP, and DXAC. Examples of antimicrobials include an antibiotic such as minocycline, clindamycin, rifampin, tigecycline, daptomycin, gentamicin, or another fluoroquinolone, an antiseptic, an antimicrobial peptide, a quaternary ammonium, or the like.

The steroid and the antimicrobial may be deposited in the polymer by a variety of techniques. In some examples, the technique used to deposit the steroid and the antimicrobial in the polymer may be selected based on characteristics of the polymer, the steroid, and/or the antimicrobial. For example, some antimicrobials may not be compatible with high temperatures, and it thus may be necessary to deposit the antimicrobial in the polymer after completing any high temperature processing steps to which the polymer is exposed.

In some examples, the polymer may be formed by curing at least one constituent component of the polymer to form a cured polymer. For example, the polymer may comprise a two part silicone, and the two parts may be mixed and allowed to cure to form the cured polymer. In such an example, the steroid and/or the antimicrobial may be mixed in at least one of the two parts prior to curing the polymer. The two parts, including the steroid and/or the antimicrobial mixed therein, may then be mixed and cured to form the cured polymer. In some examples, the uncured polymer may be cured in a mold to shape the cured polymer to a desired shape. In other examples, the uncured polymer may be cured in a different shape than is desired for composite MCRD 94, and may then be manipulated into the desired shape (e.g., by cutting the cured polymer to the shape of composite MCRD 94).

In some examples, the polymer, steroid, and/or antimicrobial may be dissolved in at least one solvent and then solvent casted to form the composite MCRD 94. For example, a biodegradable or bioabsorbable polymer, such as, for example, collagen, PLGA, PLA, PGA, PEO, POE, poly(dioxanone), a hydrophilic hydrogel, a hydrophobic hydrogel, a polyanhydride, or the like, may be dissolved in a solvent such as, for example, ethyl acetate, tetrahydrofuran, methanol, ethanol, acetonitrile, hexane, diethyl ether, chloroform, 1,4-dioxane, dichloromethane, acetone, dimethylformamide, dimethyl sulfoxide, acetic acid, or the like.

The steroid and/or antimicrobial may be dissolved in the same solvent as the polymer, or may be dissolved in one or two separate solvents. The polymer solution, the steroid solution and the antimicrobial solution then may be mixed using, for example, a static mixer, dental speed mixer, Brabender mixer, or the like. Once the mixtures have been mixed into a substantially homogeneous mixture, the substantially homogeneous mixture may be formed into a desired shape and dried to remove substantially all of the solvent. For example, the substantially homogeneous mixture maybe formed into a layer on a release liner. The layer may be formed by spray coating the substantially homogeneous mixture on the liner, air knife coating, gap coating, gravure coating, knife coating, slot die coating, metering rod coating, or the like. The formed layer of the mixture may be heated or exposed to a lower pressure to remove substantially all of the solvents from the mixture and form a polymer layer including the antimicrobial and the steroid. In other examples, the formed layer of the mixture may be freeze dried to remove substantially all of the solvents from the mixture.

In some examples, the polymer may comprise collagen. In such examples, composite MCRD 94 be formed by first creating a dispersion or suspension comprising collagen in a solvent, such as water or another non-organic solvent. The dispersion or suspension may include between approximately 0.5 weight percent (wt. %) collagen and approximately 5 wt. % collagen and a balance solvent. The dispersion or suspension then may be freeze dried to form the collagen sponge. The porosity of the collagen sponge may be influenced by the concentration of collagen in the suspension or dispersion. For example, a suspension or dispersion including a higher concentration of collagen may result in a less porous (or denser) collagen sponge. Conversely, a suspension or dispersion including a lower concentration of collagen may result in a more porous (or less dense) collagen sponge.

In examples in which a composite MCRD 94 including a denser collagen layer is desired, the collagen sponge may be formed by applying mechanical pressure and heat to a collagen sponge wetted with an amount of solvent, such as water. For example, a collagen sponge may be wetted such that the solvent content is between approximately 2 wt. % and approximately 40 wt. % of the wetted sponge. The sponge may then be exposed to a temperature between approximately 50° C. and approximately 200° C. at a pressure between approximately 0.5 kg/cm² and approximately 1000 kg/cm² for between approximately 0.1 second and approximately 1 hour to reduce porosity of the sponge and form a denser layer of collagen.

The porosity of the collagen may be influenced by the temperature, pressure, and time at which the sponge is pressed. For example, pressing the sponge at a higher pressure, at a higher temperature, and/or for a longer time may result in a composite MCRD 94 that is less porous than a composite MCRD 94 pressed at a lower pressure, a lower temperature, and/or a shorter time. Additionally or alternatively, the porosity of the collagen in composite MCRD 94 may also be affected by porosity of the initial sponge which is pressed to form composite MCRD 94. As described above, the porosity of the sponge may be influenced by a concentration of collagen in the suspension or dispersion from which the sponge is formed.

In some examples, the polymer, steroid, and antimicrobial may be mixed using milling or another high shear mixing apparatus, such as a Brabender mixer. The polymer mixed with the steroid and the antimicrobial may then be processed at an elevated temperature to form the polymer into a desired shape. For example, the polymer including the steroid and the antimicrobial may be extruded or molded at an elevated temperature. The polymer may be formed into a desired shape, which may be a sheet, disk, film, or the like. In some examples, the polymer including the steroid and the antimicrobial may be cut or stamped to the final form factor of composite MCRD 94.

Other techniques for forming composite MCRD 94 are also contemplated. For example, a polymer may be electrospun or melt blown to form a porous polymer layer. The antimicrobial and/or the steroid may then be deposited into pores of the porous polymer layer by forming a solution of the antimicrobial and/or the steroid in a solvent, introducing the solution into the pores of the porous polymer layer, and removing the solvent by drying to leave the antimicrobial and/or steroid in the pores of the polymer layer. Further details regarding method of forming a porous polymer layer with an antimicrobial disposed in pores of the porous polymer layer may be found in U.S. Provisional Patent Application Ser. No. 61/152,467, entitled, “ANTIMICROBIAL ACCESSORY INCLUDING A POROUS POLYMER LAYER,” and filed Feb. 13, 2009, which incorporated herein by reference in its entirety.

In some examples, as described above, composite MCRD 94 may include a first material and a second material. Each of the first material and the second material may be formed by any of the techniques described above, or any other suitable polymer processing technique. The first material and second material then may be coupled to one another by, for example, injection molding, compression molding, transfer molding, casting, solvent dispersion followed by casting, or the like. In some examples, the first material may be formed and then the second material may be formed on the first material by, for example, solvent dispersion followed by casting, spraying, extruding, painting, or the like. In still other examples, the first material and the second material may be coextruded to form composite MCRD 94.

In some examples, at least one of the first material and the second material may comprise collagen. In some of these examples, the first material may be coupled to the second material through use of pressure and elevated temperatures. Similar to the process described above for a composite MCRD comprising a single collagen material, the collagen, whether the first material, the second material, or both, may be wetted with a solvent, such as water, to a moisture content of between approximately 2 wt. % and approximately 40 wt. % of the wetted sponge. The first material then may be aligned with and disposed on the second material. The first and second materials may then be exposed to heat and pressure, e.g., by use of a heated press. In some examples, the first material and the second material may then be exposed to a temperature between approximately 50° C. and approximately 200° C. at a pressure between approximately 0.5 kg/cm² and approximately 1000 kg/cm² for between approximately 0.1 second and approximately 1 hour to mechanically couple the first material to the second material.

In some examples, the process of coupling the first material and the second material using heat and pressure may reduce a porosity of the first material and/or the second material. However, by appropriately selecting the pressure, temperature, and time for which the first material and second material are pressed, the extent of the porosity reduction of the first material and/or the second material may be controlled. Controlling an amount of porosity of the first material and/or the second material may influence an amount of antimicrobial or steroid with which the first materials and/or the second material may be loaded.

Once the composite MCRD 94 has been formed, the lead (e.g., lead 80 shown in FIG. 3) may be assembled from the lead components and the composite MCRD 94. For example, the composite MCRD 94 may be disposed in a cavity in electrode tip 96. Conductors 86 may be disposed within outer insulative sheath 84 and respective conductors 86 may be connected to first electrode 88 and second electrode 90. Electrode tip 96 may be coupled to lead body 82 (e.g., outer insulative sheath 84). The assembled lead 80 may then be sterilized by, for example, electron beam, gamma beam, ethylene oxide, autoclaving, or the like.

Various examples have been described in the disclosure. These and other examples are within the scope of the following claims.

1. A lead comprising:
a lead body comprising a proximal end and a distal end;
an electrode formed proximate to the distal end; and
a monolithic controlled release device located proximate to the electrode, the monolithic controlled release device comprising:
a polymer;
a steroid mixed in the polymer; and
an antimicrobial mixed in the polymer.

2. The lead of claim 1, wherein the polymer is a first polymer, and wherein the monolithic controlled release device further comprises:
a first material comprising the first polymer and the steroid mixed in the first polymer; and
a second material comprising a second polymer and the antimicrobial mixed in the second polymer.

3. The lead of claim 2, wherein the first polymer comprises at least one of silicone or polyurethane.

4. The lead of claim 2, wherein the second polymer comprises at least one of silicone, polyurethane, collagen, poly(lactic-co-glycolic acid), poly(lactic acid), poly(glycolic acid), poly(ethylene oxide), poly(ortho ester), poly(ε-caprolactone), poly(dioxanone), polyglyconate, hyaluronic acid, gelatin, fibrin, fibrinogen, cellulose, starch, cellulose acetate, polypyrrolidone, a poly(ethylene oxide)/poly(propylene oxide) copolymer, poly(ethylene vinyl acetate), poly(hydroxybutyrate-covalerate), polyanhydride, poly(glycolic acid-co-trimethylene carbonate), polyphosphoester, polyphosphoester urethane, a poly(amino acid), a cyanoacrylate, poly(trimethylene carbonate), poly(iminocarbonate), a copoly(ether-ester) such as poly(ethylene oxide)/poly(lactic acid), a polyalkylene oxalate, a polyphasphazene, a polyarylate, a polyacrylate, poly(vinyl alcohol), poly(vinyl acetate), carboxymethyl cellulose, poly(acrylic acid), or a sugar ester.

5. The lead of claim 2, wherein the first material and the second material comprise the same polymer.

6. The lead of claim 1, wherein the antimicrobial comprises at least one of a tetracycline, a rifamycin, a macrolide, a penicillin, a cephalosporin, a beta-lactam antibiotic, an aminoglycoside, a glycopeptide, a quinolone, afusidic acid, trimethoprim, metronidazole, mupirocin, a polene, an azole, a beta-lactam inhibitor, tigecycline, daptomycin, clindamycin, or another fluoroquinolone, an antiseptic, an antimicrobial peptide, or a quaternary ammonium.

7. The lead of claim 6, wherein the antimicrobial comprises gentamicin.

8. The lead of claim 1, wherein the steroid comprises at least one of beclamethasone, dexamethasone, dexamethasone sodium phosphate, or dexamethasone acetate.

9. The lead of claim 1, wherein the monolithic controlled release device is positioned within approximately 10 mm of the electrode.

10. The lead of claim 1, further comprising a fixation element located proximate to the electrode and the monolithic controlled release device.

11. The lead of claim 10, wherein the electrode comprises a helical tip electrode, and wherein the helical tip electrode comprises the fixation element.

12. The lead of claim 10, wherein the monolithic controlled release device is positioned within approximately 10 mm of the fixation element.

13. A system comprising:
an implantable medical device; and
a lead coupled to the implantable medical device, the lead comprising:
a lead body comprising a proximal end and a distal end;
an electrode proximate to the distal end of the lead body; and
a monolithic controlled release device located proximate to the electrode, the monolithic controlled release device comprising:
a polymer,
a steroid mixed in the polymer, and
an antimicrobial mixed in the polymer.

14. The system of claim 13, wherein the polymer is a first polymer, and wherein the monolithic controlled release device further comprises:
a first material comprising the first polymer and the steroid mixed in the first polymer; and
a second material comprising a second polymer and the antimicrobial mixed in the second polymer.

15. The system of claim 14, wherein the first polymer comprises at least one of silicone or polyurethane.

16. The system of claim 14, wherein the second polymer comprises at least one of silicone, polyurethane, collagen, poly(lactic-co-glycolic acid), poly(lactic acid), poly(glycolic acid), poly(ethylene oxide), poly(ortho ester), poly(ε-caprolactone), poly(dioxanone), polyglyconate, hyaluronic acid, gelatin, fibrin, fibrinogen, cellulose, starch, cellulose acetate, polypyrrolidone, a poly(ethylene oxide)/poly(propylene oxide) copolymer, poly(ethylene vinyl acetate), poly(hydroxybutyrate-covalerate), polyanhydride, poly(glycolic acid-co-trimethylene carbonate), polyphosphoester, polyphosphoester urethane, a poly(amino acid), a cyanoacrylate, poly(trimethylene carbonate), poly(iminocarbonate), a copoly(ether-ester) such as poly(ethylene oxide)/poly(lactic acid), a polyalkylene oxalate, a polyphasphazene, a polyarylate, a polyacrylate, poly(vinyl alcohol), poly(vinyl acetate), carboxymethyl cellulose, poly(acrylic acid), or a sugar ester.

17. The system of claim 14, wherein the first material and the second material comprise the same polymer.

18. The system of claim 13, wherein the antimicrobial comprises at least one of tetracycline, a rifamycin, a macrolide, a penicillin, a cephalosporin, a beta-lactam antibiotic, an aminoglycoside, a glycopeptide, a quinolone, afusidic acid, trimethoprim, metronidazole, mupirocin, a polene, an azole, a beta-lactam inhibitor, tigecycline, daptomycin, clindamycin, or another fluoroquinolone, an antiseptic, an antimicrobial peptide, or a quaternary ammonium.

19. The system of claim 18, wherein the antimicrobial comprises gentamicin.

20. The system of claim 13, wherein the steroid comprises at least one of beclamethasone, dexamethasone, dexamethasone sodium phosphate, or dexamethasone acetate.

21. The system of claim 13, wherein the monolithic controlled release device is positioned within approximately 10 mm of the electrode.

22. The system of claim 13, wherein the lead further comprises a fixation element located proximate to the electrode and the monolithic controlled release device.

23. The system of claim 22, wherein the electrode comprises a helical tip electrode, and wherein the helical tip electrode comprises the fixation element.

24. The system of claim 22, wherein the monolithic controlled release device is positioned within approximately 10 mm of the fixation element.

25. The system of claim 13, wherein the implantable medical device comprises at least one of a pacemaker, a cardioverter, a defibrillator, a drug delivery device, a monitoring device, a neurostimulator a cardiac lead, a neurological lead, a catheter, or an orthopedic device.

26. A method comprising:
forming a monolithic controlled release device (MCRD) comprising a polymer, a steroid, and an antimicrobial;
assembling the MCRD, a lead body, a conductor, and an electrode to form a lead in which the MCRD is proximate to the electrode.

27. The method of claim 26, wherein the polymer comprises a first polymer, and wherein forming the monolithic controlled release device comprises:
forming a first material comprising the first polymer and the steroid;
forming a second material comprising a second polymer and the antimicrobial; and
coupling the first material and the second material.

28. A method comprising:
implanting in a patient a lead comprising a lead body including a proximal end and a distal end, an electrode formed proximate to the distal end, and a monolithic controlled release device (MCRD) located proximate to the electrode, wherein the MCRD comprises a polymer, a steroid mixed in the polymer and an antimicrobial mixed in the polymer;
eluting the steroid from the MCRD to a tissue of the patient proximate to the MCRD; and
eluting the antimicrobial from the MCRD to the tissue of the patient proximate to the MCRD.
